(19) United States
(12) Patent Application Publication
(10) Pub. No.: US 2011/0201121 A1
(43) Pub. Date: Aug. 18, 2011

(54) METHOD OF TESTING A LIQUID SAMPLE, A TEST UNIT, AND AN AUTOMATIZED SYSTEM OF A PLURALITY OF TEST UNITS

(75) Inventor: Niilo Kaartinen, Kuusisto (FI)

(73) Assignee: BERGGREN OY AB, Helsinki (FI)

(21) Appl. No.: 12/438,621

(22) PCT Filed: Aug. 24, 2006
(86) PCT No.: PCT/FI2006/000282

§ 371 Date: Mar. 19, 2009

Related U.S. Application Data
(60) Provisional application No. 60/710,492, filed on Aug. 24, 2005.

(51) Int. Cl. G01N 33/00; G01N 35/00

(52) U.S. Cl. 436/43; 422/68.1

(57) ABSTRACT

A method of testing a liquid sample, a test unit, an and automatized system of a plurality of test units. The system is for large scale testing patient blood samples, comprising a large number of test units at different locations and connected through a common control unit. In an individual test unit there is a liquid dosing chamber, hermetically closed packages of calibrator and control liquids, closed liquid reagent packages, a liquid sample inlet and an actuator for dosing and mixing the reacting components and elements for registering results from the reactions. The test unit can calibrate, control and test reactions automatically under surveillance of the control unit. The system can use the same packaged liquids in each of the test units, enabling calculation of averages of the control results and, through comparison of individual results with an average, letting faults be detected through results outside permitted deviations.

The invention relates to a method of testing a liquid sample, especially in the field of biochemical and clinical chemistry to test blood-based samples derived from patients for various diseases or other health conditions. Further objects of the invention are a test unit for carrying out the method of the invention, as well as a system comprising a plurality of test units at various locations subjected to centralized automatic operation through a data transmitting network of communications.

The general aim of the invention is to improve quality control of chemical, biochemical and clinical laboratory testing. The most frequent testing type is known as In-Vitro-Diagnostics (IVD). Most IVD tests are performed on the serum or plasma, derived from the bloods sample of the patient. The test results are compared to values simultaneously (˜same day) obtained from the known control serum samples. The provided control value now is typically not a single concentration but a rather wide range, reflecting the uncertainty accumulated in the traceability during the instrument manufacturing process, during manufacturing process of the control serum and in the control values of the final patient measurement. A number of factors are presently contributing to the uncertainty of the control serum product.

The general problem associated with any serum, including the control serum, is that serum is a very good medium for microbes and fungus to rapidly grow and spoil the serum whenever exposed to ambient circumstances. The present laboratory practice in the device level takes place by pipetting between open control serum cups and other analysis steps. Control serum bottles must be opened to transfer part of it as a control sample. Whenever the serum control bottle is opened to the atmosphere, it is sown with microbial and fungi contamination. Additionally there are powerful chemical effects and reactivity within serum components themselves, changing concentrations over time. Even if the opened serum bottle is stored in 2-8 degrees C. in a refrigerator, it has typically one week working time. If the serum bottle is not opened, the working time is 2 months. In a frozen state at −20 degrees the unopened the control serum preserves typically 30 months.

The classical method to extend the working time of the control serum is to preserve it in a dry form, without any liquid water, whereby a proper storage is not a problem. Freeze-drying the control serum and dividing it in so small portions that it can be used at once, the same day, is presently the most common practice. The user needs to reconstitute the control serum by adding the specified small amount of water and carefully dissolve the dry serum into a homogenous solution. The drawback of this method is that the user needs to dispense very accurately the very small amount of pure water and dissolve the content into a homogenous solution. In manufacturing there are a number of production and control steps in manufacturing small control sera in mini/micro bottles. Those production steps are monitored and process controlled so that gross errors are detected. The calibration by the end user at patient work level has least means of finding out when making gross errors in reconstitution of very small volumes. When the final user is a layman from the laboratory profession standpoint like in Point-Of-Care testing, the patient safety is jeopardized when depending on such a quality control of IVD tests. This is the reason whereby more expensive ready-to-use liquid sera provide higher quality but require a professional laboratory management.

In principle a control serum product aims to be traceable to value from a high-level reference laboratory. To accurately measure the values of all tens and hundreds of specified test compounds, requires special, tedious and so expensive reference methods and instruments, only available in few reference laboratories. Quality traceability standard ISO 17511:2003 describes steps from a reference laboratory via multiple steps in the manufacturing to the final calibration of the patient sample in the field. There are approximately ten steps. Each step is adding variation to the traceability from the reference laboratory value, totalling up to many percent as standard deviation. That is far from satisfactory from quality traceability standpoint. IVD manufacturers are transferring expensive reference calibration materials to their research laboratories, to act as a yardstick for calibrating manufacturing instruments and for calibrating controlling sera products in manufacturing. Every analyser yields a somewhat different result, between manufacturers and even within of different models of the same manufacturer.

To prevent the drift in control values between instruments and manufacturers to carry too far over time and so to jeopardize patient safety, joint external quality assurance organizations have been formed to monitor reproducibility among laboratories. The quality organization is sending the same unknown serum sample once or twice a year for each participant in the quality ring for analysis. Quality organization is statistically summarizing all test results and reports to each participant, how far from the average they are with each of their analysers, in comparison to all others having the same analyser. No stand is taken if the mean test value is right or wrong. It only tells where other laboratories are on average and how high is the standard deviation. Thus obtained feedback may take over a year, being either grossly off from others or in the middle of other laboratories using the same analyser model. In practice it is the responsibility of an attending physician to compare the laboratory IVD data to the clinical picture of the patient and not to solely trust unexpected laboratory data. Unexpected results may result from many artefact sources, starting from a faulty or wrong sample, from errors in calibration or from a spoiled reagent of the test or from analyser drift. Unexpected results indicate retesting, delays in patient care and inefficiencies in laboratory services. Origin of this uncertainty is resulting from a long chain and deteriorated traceability of testing quality, whereby detection of other sources of variation is shielded by the poor traceability and variability of calibration.

Many chemical, biochemical and clinical laboratory tests use such sensitive reagents and other analytical steps that the analyzer and the test method should be tested one or more times a day with a known test sample, called as quality control process. Traceability of quality relates to tracing back the accuracy of the quality control sample via manufacturers production and quality control processes to the values of the reference laboratory with reference methods. The present quality procedure assumes a laboratory environment and professional skills. The present practice in quality traceability contains about 10 steps, each adding variability, yielding only the range for a true value.

Working time of liquid reagents with 12-18 months are available e.g. by JAS Diagnostics, Miami, Fla. and elsewhere. Reagents for its parts would enable a full automation and remove the need of a laboratory to manage reagents. But the quality control rules require a control sample every day or even after every 8 hours, presently only well done with laboratory skills in laboratory environment. Before efficiency by automation can really advance, the problem of quality control and traceability needs to be resolved without need of professional skills.

The invention provides a solution to the problem through improvement and automatization of the quality control and quality traceability of IVD tests, performed in the test units on the field outside the laboratory, that is at Point-Of-Care sites and in other non-laboratory places without professional laboratory skills. The outcome of the invention in its wholeness is, that the quality and traceability part of IVD testing is delivered and monitored professionally but remotely all the time, so that the users can be non-professionals. Ensuring high quality of patient test data with lesser or no amount of laboratory skills is a significant advantage of the invention. Furthermore, the invention enables real time monitoring, adjusting and controlling the quality of the networked analyzers in the field using the inventive method. The invention also enables tracing and adjusting the changes in the control material, so dynamically monitoring traceability, thus extending the unattended working time of calibration from hours to months.

The method of testing a liquid sample according to the invention uses a test unit comprising a liquid dosing chamber, the method being characterized by
provision of at least one closed calibrator liquid package connected to the dosing chamber through an openable valve,
provision of at least one hermetically closed control liquid package connected to the dosing chamber through an openable valve, the control liquid differing from the calibrator liquid,
provision of at least one inlet with an openable valve for at least one liquid reagent,
provision of an inlet with an openable valve for the liquid sample to be tested,
performing at separate stages calibrating, control and test reactions between the calibrator liquid, control liquid and the liquid sample, respectively, and at least one liquid reagent,
each stage comprising opening the respective valves and sucking predetermined amounts of the reacting liquids to the dosing chamber to produce the respective reaction,
cleaning the dosing chamber between the reactions,
registering the results of the calibrating, control and test reactions, and
by comparison of said results obtaining a test result for the liquid sample.

A test unit for testing a liquid sample according to the invention is characterized in that it comprises a liquid dosing chamber,
at least one closed calibrator liquid package connected to the liquid dosing chamber through an openable valve,
at least one hermetically closed control liquid package connected to the liquid dosing chamber through an openable valve,
at least one openable inlet for at leat one liquid reagent,
an openable inlet for a liquid sample to be tested,
an actuator for sucking amounts of said calibrator liquid, said control liquid, said liquid sample and/or said at least one liquid reagent to the liquid dosing chamber at different stages, to perform calibrating, control and test reactions, respectively, and
means for registering and comparing the results of the reactions to obtain a test result.

The test unit according to the invention preferably further comprises at least one closed liquid reagent package connected to the liquid dosing chamber through an openable valve. The liquid reagent is combined in the dosing chamber with the calibrator liquid, the control liquid or the liquid sample for performing the calibrating, control or tests reactions, respectively. The liquids may be sucked to the liquid dosing chamber by means of the actuator, which may be a bellows, a pump or an injector.

Furthermore, the test unit according to the invention may comprise an openable and closable inlet for filling the liquid dosing chamber with dilution liquid. The test unit according to the invention may also comprise an openable and closable outlet for sucking dilution liquid from the liquid dosing chamber. The dosing chamber would be filled with the dilution liquid before the above-mentioned sucking operations for dosing the liquids needed for the reactions. Dosing of the reactive liquids would then take place by sucking corresponding amounts of the dilution liquid from the dosing chamber. Preferably, there is a common inlet/outlet for both the filling and sucking purposes.

As an example, the calibrating reaction performed in the test unit could include the following sequence of steps:
provision of a closed calibrator liquid package connected to a liquid dosing chamber of the test unit through an openable valve,
provision of at least one closed reagent liquid package connected to the liquid dosing chamber through an openable valve,
filling the liquid dosing chamber with dilution liquid,
sucking an amount of calibrator liquid from the package to the liquid dosing chamber by opening the respective valve and sucking a corresponding amount of dilution liquid from the liquid dosing chamber,
sucking an amount of the at least one reagent liquid from the respective package to the liquid dosing chamber by opening the respective valve and sucking a corresponding amount of dilution liquid from the liquid dosing chamber,
performing the reaction between the calibrator liquid and the at least one reagent liquid as combined in the liquid dosing chamber, and
registering the result of the reaction for use as a reference in performing the corresponding reaction with a liquid sample to be tested.

As noted, the filling and sucking steps are preferably carried out through a common inlet/outlet by means of an actuator connected therewith.

The control and test reactions could be performed in an analogous manner, the control liquid in a closed control liquid package and the test sample supplied from an inlet with a closable valve replacing the calibrator liquid, respectively.

The calibrator and control liquid packages, as well as the reagent packages, are preferably hermetically sealed collapsible bags with a diminishing volume as liquid is sucked therefrom. Such bags are described in the patent publication U.S. Pat. No. 4,588,554, which is hereby incorporated by reference in the present specification.

The control liquid used in the invention when applied to diagnostical purposes is preferably blood serum. The calibrator liquid may be a pure component of blood serum dissolved in a solvent.

The calibrator and control liquids are preferably preserved in the bags in frozen condition before use for their respective purposes in the test unit. The test unit can be equipped with a refrigerator as well as with heating means for selectively thawing the liquids to be sucked to the liquid dosing chamber.

There may be a separate reaction chamber or space connected through an openable valve with the dosing chamber. Sensors are provided either in the dosing chamber or reaction chamber or space to detect the results of the reactions.

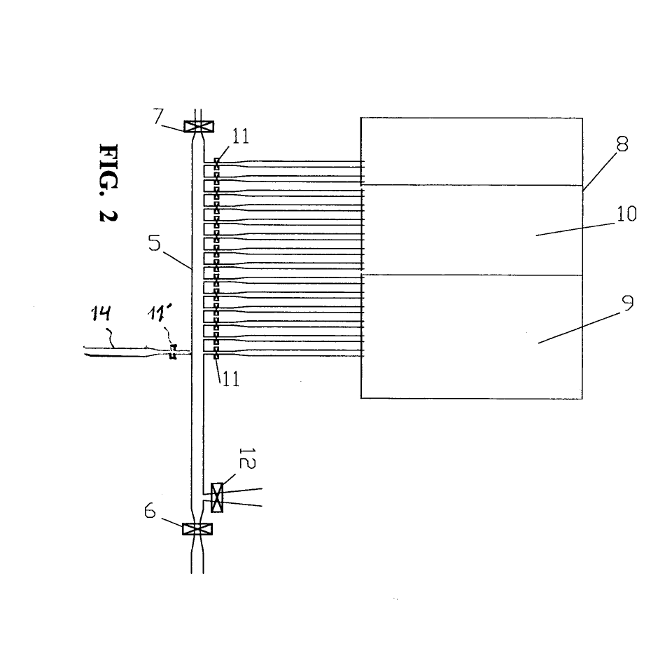

The entire test unit and its functions may generally correspond to those described in the publication WO 02/061395 A1, which is hereby incorporated by reference in the present specification. However, the reference does not disclose the combined calibration and control operations, which are essential for the present invention. The test unit of the reference comprises reagent bags designated in FIG. 2 by reference character 10, which according to the present invention would also comprise at least one bag containing a calibrator liquid and at least one bag containing a control liquid.

A system according to the invention containing a plurality of test units for testing liquid samples as defined above is characterized in that each test unit is equipped for performing the same test reaction, that the test units are differently located and each connected through data transmission communications to a common control unit monitoring the test units in the system by data transmitted through said communications, that the reaction with the control liquid is performed in each test unit at predetermined time intervals to obtain individual control values for the test units, an average of said control values of all the test units is determined, the individual control values are compared to said average, and only test units with a reference value within a permitted deviation from the average are approved for performance of the reaction with samples to be tested.

A further system according to the invention, containing a plurality of test units for testing liquid samples as defined above, is characterized in that each test unit is equipped for performing the same test reaction, that the test units are differently located and each connected through data transmission communications to a common control unit monitoring the test units in the system by data transmitted through said communications, that the performance of said test system is from time to time independently evaluated by an accurate analysis of control and calibrator liquid reference samples preserved in circumstances corresponding to those prevailing in the test units, to estimate and adjust the time effect on calibrator and on control liquids and on reagents stored in the test units.

Preferably a system according to the invention comprises both sets of features as recited above simultaneously. This is to say that calibration of the system is carried out under the direction of the control unit, the analysis of the reference samples being performed in a common laboratory, while the controls at relatively short time intervals, daily or even more frequently, are performed under automatic direction and monitoring by the control unit.

In the system each test unit has at least one accurately known calibrator liquid and/or at least one accurately known control liquid and at least one test reagent, which is the same in each test unit. Preferably the calibrator liquids, control liquids and reagents are the same, i.e. derived from the same manufacturing batches, in each test unit in the system. In such a case all the calibration and control results received from the individual test units are comparable, and averages can be calculated for each of them to be used in comparisons.

In case that in a test units a reference value outside the permitted deviation from the average is obtained, the reaction with the control liquid may be repeated at the request of the control unit one or more times to exclude the possibility of a random error, so that the control unit can approve previous and subsequent test results.

The method of the invention, the test unit using the method, and the quality monitoring system comprising said test units can be summarized as follows:

1. The method comprises use of liquid control serum without ever functionally opening its package and so not shortening its working time when virtually preserved as unopened.

2. The basic device to exercise the invention corresponds functionally to a hermetic valve connecting the hermetic calibrator serum package to the liquid processing part, i.e. a dosing or reaction chamber, of an analyzer.

3. The system comprises monitoring the said inventive calibration devices, i.e. the test unit, and calibration functions in the field via a network, working in real time from the calibration standpoint and secures that only proper values are used even when the calibration values of the used control sera are changing over time.

This invention eliminates variability effects and a short working time of manual handling and exposing control sera and calibrators to ambient atmosphere, in connection of testing patient samples. The virtual unopenness for a long working time and hydraulic accuracy and reliability in key liquid processing enables full automation so that a plurality of test units, all loaded with the same, known high quality control sera, calibrators and reagents, can be networked under a common control unit, for an automatic co-monitoring the quality data. When an error is detected, repeated readings of control tests are automatically performed to exclude random errors. If repeated controls can not resolve the detected problem, then the same test is automatically performed with a stable calibrator. If the result is the same as before, control serum is bad. If the test result is changed and the controlserum test is proportionally changed, the control serum is good and the test reagent is bad. Furthermore, the automatic co-monitoring of the common control unit allows an automatic, real time, high precision evaluation of a true stability or drift sera and of control reagents as a standard error of means of test data from plurality of test units. The high precision of measured average values from a plurality of test units allows more reliable testing than gives a short term, nominally stable control or calibrator with given wide error margins. As summary, the major cost and quality benefit of the invention is, that the errors, time delays and costs relating manual laboratory work is eliminated and the statistical reliability from real time quality related data from plurality of test units provides superior security in quality.

A more detailed description of the various aspects and steps of the preferred embodiment of the invention is:
1. Calibration of a large lot of control serum at a high accuracy level
2. Packaging the serum in hermetic, air-free, collapsible packages, e.g. such as described in U.S. Pat. No. 4,588,554, so that liquid can be taken out without anything going in and freezing the packages for longer term storage. Further, a sterile filtering in filling, removing air or oxygen will reduce spoiling.
3. Installing one or more frozen control serum packages into a test unit to cover the capacity and working time of the rest of the system. One of the packages is automatically melted and made available in a hermetic manner for automatic analysis for the capacity or until the expiration date as unopened. Other packages are retained as frozen.
4. The calibrating means comprise a functionally hermetic valve connecting the hermetic control serum package and the liquid dosing system. Functionally hermetic means that nothing can enter the hermetic calibrator package, the only flow direction is out. Technically it can be ensured in a number of ways. A check valve prevents the backflow and access. A positive pressure head from the control package ensures a leak out if any. A closed, hydraulic sampling, e.g. as is used in HPLC or as described in pre-grant publication US 20040115829 (corresponds to WO 02/061395 A1), would provide much higher precision than the pipetting between open liquid surfaces. The benefit is that the control serum package is functionally unopened and so would maintain its present working time 2 months, which can be extended with the system described below. Multiple frozen packages could be installed within a test unit, having automatic means of connecting, freezing and thawing to maintain really long, unattained operation. Since the liquid storing temperature is critical, temperature stability is monitored and its history is collected for monitoring. This would enable full automatic analyzers, “Black-Boxes” (BB), to be used by laymen outside the laboratory.
5. BB-analyzers having the calibration means installed, could also provided (inter)net connection to centrally monitor all calibrating result values, done typically in the first most quiet hours of each day. If any of the calibrating result values of any test within any test unit is outside the proper values, the test in that test unit can be centrally turned off or properly flagged before a work shift starts. It does not matter if the error is coming from the calibrator liquid, from reagents or from instrument hardware or from software. The statistical real time information from the large pool of test units gives a good statistical tool to handle single exceptional events at the field automatically via a database.
6. In an otherwise good liquid control serum may some components loose activity over time and some other components maybe increased from bound to active state. To extend the working time of a control serum lots in the test units in the field, lots are carefully monitored centrally for these and other possible small changes in exactly the same refrigerated conditions than the calibrator means operate in the field. Any such small but systematic changes in control values would over time—as uncorrected—lead to wrong calibration. The central monitoring the control values is then updating automatically calibrator values in the field for tests and lots concerned. The working life of calibrators would be extended, possibly even greatly.
7. When a calibrator result value of a test is that way ensured and a test unit in the field provides a calibrator value outside the proper range, a “fault” value, it means that reagents are not working properly because electronic and other measuring steps are internally controlled and reported OK. If not, the test unit is automatically replaced in the field. This way the whole network of test units are successfully monitored, serviced via network by readjustments or replaced without user's participation.

The test unit according to the invention is described in the following in more detail with reference to the drawings, in which

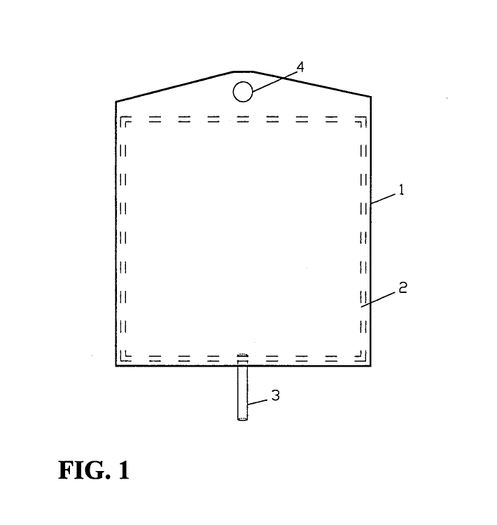

FIG. 1 shows a liquid package in the form of a collapsible bag applicable in the invention,

FIG. 2 shows an embodiment of the test unit according to the invention,

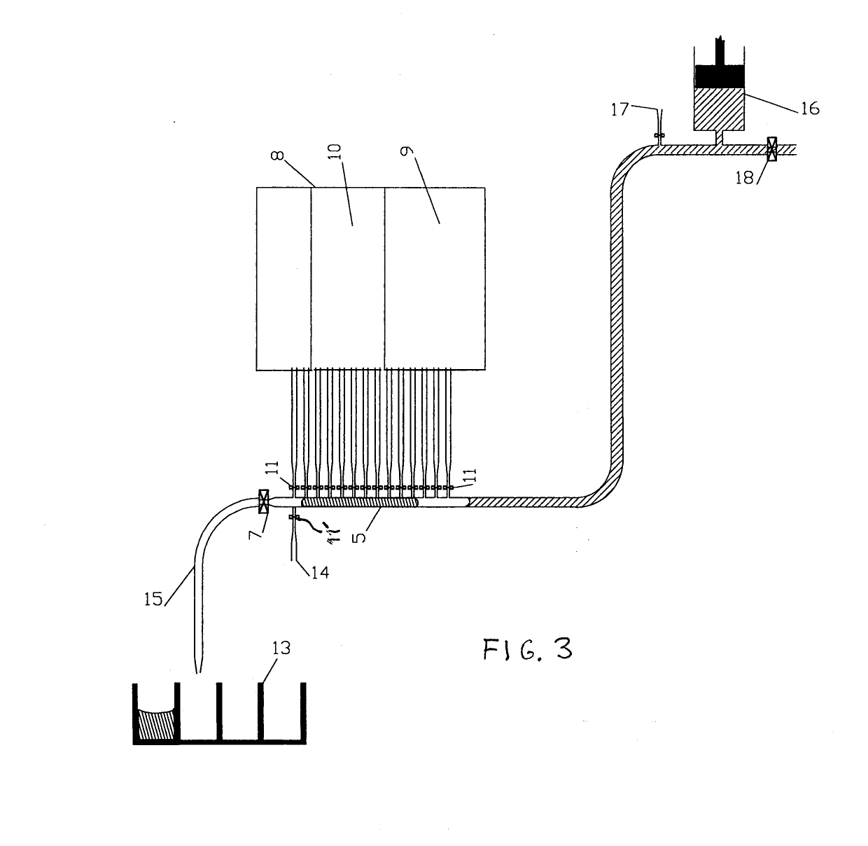

FIG. 3 shows another test unit according to the invention, and

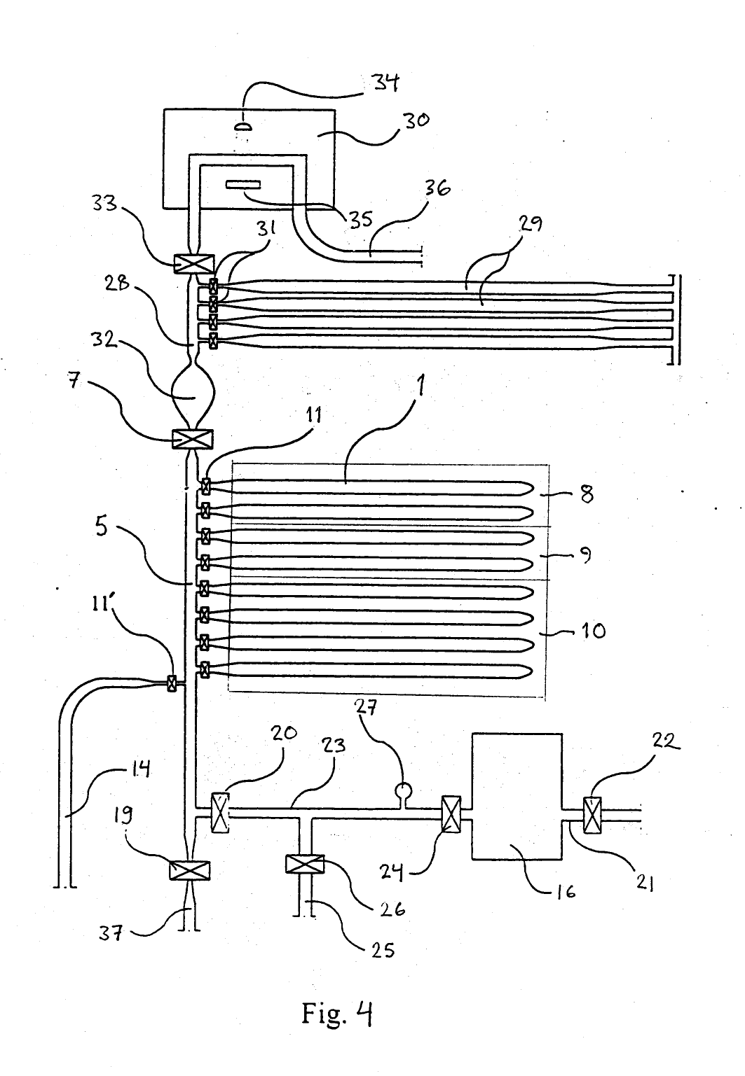

FIG. 4 shows still another test unit according to the invention.

The invention relates to a test system in clinical chemistry, which conducts diagnostic tests of liquid samples from a patient, such as blood samples. The system consists of a central control unit, which preferably communicates over wireless telecommunications with a multitude of test units remote relative to the control unit. The test units acting as remote terminals in the system carry out practical tests under the control and surveillance of the central control unit.

In FIG. 1 there is described as an example a collapsible hermetic liquid package 1 in the form of a bag, the liquid being a calibrator liquid, a control liquid or a reagent. The control liquid is for instance blood serum, and the calibrator liquid is a reactive component of blood serum. Other collapsible, sufficiently hermetic bags exist on the market. Such a bag can consist of two sandwich foils having polyester to outside, aluminum foil in the middle and polyethylene inside. The two sandwich foils are heat sealed along lines 2 joining inner polyethylene layers. A heat sealable connecting tube 3 connects the content of the bag 1 to liquid processing system. The top of the package 1 has an opening 4 for hanging the filled bag so that there is a pressure head towards tube 3 and towards the liquid processing system of the test unit situating below. Bags can be sterilized and evacuated before filling with control serums via a tube 3 and after filling hermetically closed for storing and freezing before long term storing.

FIG. 2 shows a test unit providing a hermetic access to refrigerated calibrator and control liquids. The unit as shown only comprises the liquid processing section. The rest of the test unit, like electronics, detection, results calculation may be the same as in semiautomatic analyzers, the most frequent analyzer in the clinical laboratory. The section processing liquids is very simple. It has a liquid dosing chamber 5 in the form of an elongated channel closable with valves 6 and 7. Above that is a refrigerated liquid storage containing numerous liquid packages 1. There is at least one calibrator liquid package in reservoir 8, at least one control liquid package in reservoir 9 having working life at least 2 months in refrigerated liquid, and a number of reagent packages in reservoir 10 preserving up to two years, as well as other liquid species needed for testing similarly packaged in reservoir 10. The reservoir 9 contains an arrangement keeping one control liquid, such as blood serum, bag in a freezer temperature and the others in deep-freezer temperature with programmable, addressable thawing, when the time or the capacity of the working bag is expiring. The on-line access to liquid dosing chamber 5 takes place via normally hermetically closed valves 11. Valve 12 provides access to an actuator, which is a high precision liquid dispenser, filling hydraulically with pure water the chamber 5 between valves 6 and 7. When no air is present, reagents and control serum can be drawn within the diluent with very high precision of one nanoliter reproducibility. An inlet conduit 14 for the liquid sample to be tested is connected to the dosing chamber 5 through a valve 11′, working like the other valves 11. It is clear that when reagents and calibrator and control liquids are boxed in the closed test unit, calibration and controls can be initiated automatically without any manual or local operation as programmed e.g. once a day or remotely. Any other valve system, providing high precision and being otherwise suitable for analytical work, could well be used, too. When all key liquids determining the quality of analysis can be loaded once or twice a year, it can well be done by professional analyzer service engineers in connection of typical twice a year service visits. When the liquid accuracy is 20 times higher in the described test unit than in laboratory analyzers, consumption and size of a test unit according to the invention can be made correspondingly smaller. Test units with calibration and control means may be portable or transportable, whereby once or twice a year servicing can take place centrally by experts. When liquid management is taken care by analyzing personnel taking trips to service stations and monitoring of quality takes place remotely by databases and by professionals, laboratory contributing ⅘ of all IVD costs is redundant.

In FIG. 3 this invention is applied to a typical clinical analyzer with only relevant parts of the test unit schematically shown. Analysis takes place in integrated or individual reaction/measuring cells 13, where doses of calibrator and control liquids as well as test samples are respectively dispensed together with the reagents before mixing, incubation and measurement. The outlet conduit from the valve 7 forms the dispensing probe 15 to deliver precisely measured control or calibrator liquids to the measuring cell 13. Calibrator or control liquid is selected by a proper valve within valve arrays 11, connecting to hermetic test material reservoirs 8-10. For the precise dosing the outlet valve 7 is closed and an actuator 16, like a syringe as shown or a peristaltic pump, is drawing e.g. under a pressure feed back control the dose in chamber 5 and then immediately closing the open valve in valve array 11. The sample inlet conduit 14 and valve 11′ open to the dosing chamber 5 like any of the valves 11. Valve 18 provides connection to water, used as diluent and for cleaning, to fill the syringe 16. Valve 17 is opened to air for picking air buffers when needed. Water as a system liquid is used to transmit the volumetric changes of the actuator 16. An air buffer is typically arranged between the system water and control/calibrator dose, or a sample dose from conduit 14. The highest precision in dosing is achieved if the dosing chamber 5 is hydraulically filled to the outlet valve 7 but then a rather large amount of water diluent must be dispensed together with the calibrator or control liquid or test sample because without air buffer boundary liquids “telescope” inside each other in a laminar flow. The liquid material reservoirs 8-10 may contain also reagents, but more typical in a laboratory analyzer is that reagents, which form the bulk of testing volume and typically preserve long even when opened to atmosphere, are dispensed separately to the dosing chamber 5, or to measuring cells 13 then constituting dosing spaces, by use of larger dispensers. Then the actuator 16 can be selected for smaller volumes and for higher precision. When dosing is satisfactory done, valve 7 is opened and the liquid dose is pushed up or with the air buffer. For a higher precision part of a known amount of water may also dosed to the measuring cell 13 to carry the liquid film left behind by air buffer. Any reagents present in material reservoirs 8-10 are dosed together with the calibrator or control liquid or with the test sample.

Inventive benefits realize when the control and calibrator liquid packages are not opened to atmosphere and the dosing takes place in a closed system where pressure feed back from dosing operations are feasible, contrary to dosing among open vessels, what contaminate and at the absence of feedback dosing operations are “blind”. Precision determining control liquid such as control serum thus preserve long on-board and so the manual, variability adding laboratory work with control sera is completely avoided. Automatic analysis based on closed, feed back controlled precision dispensing of long preserving control materials allows remote, networked co-monitoring of control values of a large number of test units according to the invention. The database analysis provide high precision information of the actual real time working values of control materials and so also of reagents, without trusting and assuming the nominal short term stable values manufacturers of control sera are providing in the compulsory labelling.

FIG. 4 shows the liquid processing parts of a test unit, which makes a diagnostic test of a liquid medical specimen, such as a blood sample taken from a patient, by carrying out a test reaction and by detecting the end result of the reaction. The test unit also performs the necessary calibrating and control reactions and detects the results in a similar manner. The test unit comprises an elongated, tubular dosing chamber 5, to which the needle-like suction duct 14 forming the sample inlet is connected through valve 11′. In addition, the dosing chamber 5 is connected with a plurality of liquid packages 1 in liquid storage reservoirs 8-10 through valves 11, there being a reservoir 8 for at least one calibrator liquid, a reservoir 9 for at least one control liquid, and a reservoir 10 for reagents. The reagents needed in the test reactions and calibration and control reactions to be performed with the test unit are stored in the reagent bags in reservoir 10. The test unit is provided with a cooler (not shown) for keeping at least part of the calibrator liquid, control liquid and reagent packages it their respective reservoirs 8-10 freezed, as well as means for selective thawing of the packages as the liquid is needed for the reactions. The feed duct 14 and the connections to each liquid package are equipped with an on/off valve 11, 11′, and in addition, the dosing chamber 5 is defined by on/off valves 7, 19, 20. The valves thus determine the volume of the dosing chamber 5. For dilution of the sample to be tested and the reagents required for the test, the test unit uses water as the medium, and for this purpose the unit is connected to the water mains over a pipe 21 equipped with a valve 22. Water is supplied from the pipe 21 into a reciprocating bellows 16 acting as an actuator, which communicates over a duct 23 equipped with valves 20, 24 with the dosing chamber 5. The test unit is devised so as to control liquid transfer in all the parts of the unit with suction and expulsion movements generated by the bellows 16.

An air duct 25 equipped with a valve 26 is connected to said duct 23 between the dosing chamber 5 and the bellows 16. This allows the use of air as a buffer for the transfer of liquids from one part to the other of the test unit. The duct 23 is additionally equipped with a pressure sensor 27 acting as a sensor, allowing the monitoring of liquid movements generated with the bellows 16 in different parts of the unit. The sensor 27 detects each starting, arrival to the valve location and stop of the liquid as a pressure variation in the duct system.

At the end opposite to the bellows 16 and to the water and air inlets 21, 25, the dosing chamber 5 ends in a valve 7, from where a duct 28 continues to the incubation spaces 29 and the detection space 30 formed of a measuring filter trough in the test unit. Adjacent incubation spaces 29 are separated from the duct 28 with on/off valves 31, and the reaction mixture, which is formed in the dosing chamber 5 and consists of the sample to be tested in these, one or more reagents and water acting as a medium can be conserved over the period required for the rest reaction at a regulated reaction temperature. For mixing associated liquids in the dosing chamber 5, the duct 28 comprises an expansion 32 acting as a mixing chamber. The aligned connections between the incubation spaces 29 are followed by an on/off valve 33 in the duct 28, preceding the measuring filter trough 30, which is equipped with a light source 34 and a detector 35. An outlet duct 36 continues from the measuring filter trough 30 for discharging the liquid used for the test from the test unit.

The parts of the test unit used for dosage, calibration, controls, test reaction and result detection need to be cleaned between the tests, and for this purpose the block is equipped with a detergent liquid inlet 37, which is connected to the dosing chamber 5 and separated from this with a valve 7. The detergent liquid can be discharged from the block into an outlet duct 36 starting from the measuring filter trough 30.

In the initial situation of the test to be conducted with the test unit, the liquid treatment parts are cleaned and dried, and the valves 7, 11 and 19 defining the dosing chamber 5 are closed. The user of the unit connects the needle-like suction duct 14 acting as the sample inlet to the sample to be examined and starts the process. The bellows 16 then draws sample in the duct 14 all the way to the valve 11′, and then the valve 11′ is closed.

Next, the bellows 16 carries out filling of the dosing chamber 5 with water acting as a medium, supplied from the water pipe 21. The valve 7 after the dosing chamber 5 is opened, and the bellows 16 propels water through the duct 23 into the dosing chamber until the water reaches the opened valve 7. Water entering the valve gap entails a small change of pressure, which is recorded by the pressure sensor 27, and at that moment the automation closes the valve 7. As the filling starts, the valves 26 and 19 of the air and detergent liquid inlets 25, 37 are also open, so that water propelled by the bellows 16 fills these pipes all the way to the valves and the pressure sensor 27 causes the valves to be closed at the moment of filling. As a result of these operations, the dosing chamber 5 is hydraulically filled with water.

At the subsequent sample dosing stage, the valve 11′ of the sample inlet 14 is opened, and the bellows 16 draws water from the dosing chamber 5, so that an amount of sample equalling the exhaust suction is sucked from the inlet 14 into the dosing chamber. This sample dosing stage ends when the valve 11′ of the inlet 14 is closed.

For calibration, calibrator liquid is similarly sucked from a liquid package in reservoir 8, by opening the respective valve 11, sucking an amount of calibrator liquid by means of the bellows 16 to the liquid dosing chamber 5, and then closing the valve. For control, an amount of control liquid is likewise sucked from a liquid package in reservoir 9 to the liquid dosing chamber 5, by the valve opening, sucking and valve closing steps. The calibration and control reactions are then carried out in the same way as the sample testing, which is described in detail in the following.

Next, one or more reagents needed in the test is dosed accordingly into the dosing chamber 5. The valve 11 closing the package in the reservoir 10 containing the selected reagent is opened, and the bellows 16 draws water from the dosing chamber 5 so that an amount of water equalling the exhaust suction is drawn into the dosing chamber, after which the valve 11 is closed. If more than one reagent is needed, the dosage of the different reagents is carried out one by one with the operations described above. As a result, the dosed sample and the reagents are brought into the elongated, tubular dosing chamber 5 in succession, without notable mixing of the liquids at this stage. When the dosage is ended, the valve 20 of the dosing chamber 5 facing the bellows is closed and the duct 23 is purged of water by absorption with the bellows 16.

The following stage of the test comprises mixing of the dosed liquids and mutual reacting of the sample and the one or more reagents. To this end, the valve 7 after the dosing chamber 5 and the valve 31 connected with the selected incubation space 29 are opened, and the dosed liquids are expelled with the bellows from the chamber 5 into the mixing chamber 32, and in conjunction with this, the valve 26 of the inlet 25 is opened in order to use air as a buffer for the liquid transfers produced by the bellows. The liquids are mixed in the chamber 32 with reciprocating movements generated by the bellows, and part of the mixed liquid is further expelled with the bellows to the measuring filter trough 30 for determination of the initial value of measurement. The main portion of the liquid is propelled with the bellows into the incubation space 29 over the period needed for the test reaction to develop. While the reaction is taking place, the measuring filter trough 30 is cleaned with the detergent liquid supplied from the pipe 37, the detergent being propelled through the dosing and mixing chambers 5, 32 to the measuring filter trough and further to the outlet duct 36 by means of air supplied from the pipe 25. After the reaction, the reaction mixture is drawn from the incubation space 29 to the duct 28 between the dosage space and the measuring filter trough and is expelled into the filter trough 30 for final measurement. After the measurement, the reaction mixture is expelled by air into the outlet duct 36.

The removal of the reaction mixture after the test from the test unit may be integrated in the operation of cleaning the unit between the tests. The detergent liquid is supplied from the pipe 37 and is driven by the bellows 16 with air conducted from the pipe 25 through the dosing chamber 5, the mixing chamber 32 and the measuring filter trough 30 to the outlet duct 36. Air flows in the centre of the chambers and the ducts, and a very small amount of water is enough for cleaning in the form of an air-driven film along the walls of the spaces and the films. The sample inlet 14 is cleaned by opening the valve 11′, so that air propelled by the bellows drives detergent liquid through the inlet 14 out from the liquid test unit.

All of the liquid treatment operations described above take place under automatic control of the test unit, which is sealed from the environment with the exception of said inlets 14, 21, 25 and 37. The result can be transmitted for processing in the central control unit as automatic data transmission.

In the test unit described above, all the dosages are hydraulic precision dosages owing to the repetition precision of the movements of the bellows 16, the dosages being ensured by means of the pressure sensor 27. The reagents contained in the reagent bags are concentrated storage solutions, the central control unit being continuously informed of the remaining amounts of reagents. When purged, the reagent and other liquid bags collapse without requiring replacement air, and their uncontaminated conservation is ensured in the closed block. By means of automation, it can be continually ensured that a vacuum generated by the bellows 16 is prevailing continually while the valves 11 are open, so that the liquids are merely allowed to flow out from the bags into the dosage space, and never in the opposite direction.

A system comprising a plurality of test units as described above constitutes a network, in which the individual test units are connected through the central control unit. Each test unit has stored in the reservoirs the same calibrator and control liquids and the same reagents. As the number of test units on the field is large, benefits are gained through production of the various liquids and reagents in large batches with reduced manufacturing costs.

Furthermore, the liquids and reagents being the same, the results from various units can be compared, and the control unit will calculate averages for the results of the calibration and control reactions as received from the test units. In this way any errors or anomalies in the results from any test unit can be instantly recognized, and necessary measures can be taken, either as repeated control reactions to detect the origin of the error or shutting the faulty test unit out of operation.

In general the calibration reactions need be performed in the test units only occasionally, particularly when new batches of the various liquids are taken into use. The control reactions instead are required at frequent intervals, advantageously every morning before the tests with patient samples are started, or even every 8 hours for instance. Each time the result of the control reaction is compared to the average of all the test units in the system, to ensure that the unit is in order and can be used for tests.

In case an error in an individual test unit is detected or suspected, both the calibration and the control reactions are made in the unit. If the result of these two reactions is the same, it reveals that a reagent is spoiled, whereas if the results are different it means that the control liquid is spoiled. All these reactions are carried out in the test unit automatically under the surveillance of the control unit, and the only corrective measure to be made manually is to replace the faulty liquid package.

It is obvious to those skilled in the art that the embodiments of the invention are not confined to the example described in detail above, but may vary within the scope of the following claims.

1. A method of testing a liquid sample in a test unit comprising a liquid dosing chamber, characterized by
provision of at least one closed calibrator liquid package connected to the dosing chamber through an openable valve,
provision of at least one hermetically closed control liquid package connected to the dosing chamber through an openable valve, the control liquid differing from the calibrator liquid,
provision of at least one inlet with an openable valve for at least one liquid reagent,
provision of an inlet with an openable valve for the liquid sample to be tested,
performing at separate stages calibrating, control and test reactions between the calibrator liquid, control liquid and the liquid sample, respectively, and at least one liquid reagent,
each stage comprising opening the respective valves and sucking predetermined amounts of the reacting liquids to the dosing chamber to produce the respective reaction,
cleaning the dosing chamber between the reactions,
registering the results of the calibrating, control and test reactions, and
by comparison of said results obtaining a test result for the liquid sample.

2. A method according to claim 1, wherein there is provided at least one closed liquid reagent package connected to the dosing chamber through an inlet with an openable valve.

3. A method according to claim 1, wherein at each stage before the sucking operations the dosing chamber is filled with dilution liquid.

4. A method according to claim 3, wherein said sucking operations are effected by sucking a corresponding amount of dilution liquid from the dosing chamber.

5. A method according to claim 4, wherein filling the liquid dosing chamber with dilution liquid and sucking dilution liquid from the liquid dosing chamber are performed through a common inlet/outlet connection.

6. A method according to claim 5, wherein said filling and sucking operations are carried out with an actuator acting on the inlet/outlet connection.

7. A method according to claim 6, wherein said actuator is a bellows, a pump or an injector.

8. A method according to claim 1, wherein there is a reaction chamber connected with the dosing chamber through an openable valve, said valve being opened, and the reacting liquids sucked to the dosing chamber being respectively transferred to the reaction chamber.

9. A method according to claim 1, wherein the calibrator liquid package is a sealed collapsible bag with a diminishing volume as liquid is sucked therefrom.

10. A method according to claim 1, wherein the control liquid package is a hermetically sealed collapsible bag with a diminishing volume as liquid is sucked therefrom.

11. A method according to claim 1, wherein the at least one reagent package is a sealed collapsible bag with a diminishing volume as liquid is sucked therefrom.

12. A method according to claim 1, wherein the control liquid is preserved in frozen condition and thawed before it is sucked to the dosing chamber.

13. A method according to claim 1, wherein the calibrator liquid is preserved in frozen condition and thawed before it is sucked to the dosing chamber.

14. A method according to claim 1, wherein the control liquid is blood serum, and the liquid samples tested are blood-based.

15. A method according to claim 1, wherein calibrator and/or control liquid reference samples are preserved in circumstances corresponding to those prevailing in the test unit and analyzed from time to time, to estimate the condition of a calibrator and/or control liquid stored in the test unit.

16. A method according to claim 1, wherein the calibrating reaction is performed as a new calibrator or control liquid or reagent package is installed in the test unit.

17. A method according to claim 1, wherein the control reaction is performed at regular intervals, preferably at least daily.

18. A test unit for testing a liquid sample, characterized in that it comprises
a liquid dosing chamber,
at least one closed calibrator liquid package connected to the liquid dosing chamber through an openable valve,
at least one hermetically closed control liquid package connected to the liquid dosing chamber through an openable valve,
at least one openable inlet for at least one liquid reagent,
an openable inlet for a liquid sample to be tested,
an actuator for sucking amounts of said calibrator liquid, said control liquid, said liquid sample and/or said at least one liquid reagent to the liquid dosing chamber at different stages, to perform calibrating, control and test reactions, respectively, and
means for registering and comparing the results of the reactions to obtain a test result.

19. A test unit according to claim 18, characterized in that it comprises at least one closed liquid reagent package connected to the liquid dosing chamber through an inlet with an openable valve, for combining at least one liquid reagent with the calibrator liquid, the control liquid and the liquid sample, respectively, in the dosing chamber.

20. A test unit according to claim 18, characterized in that it further comprises an openable and closable inlet for filling the liquid dosing chamber with dilution liquid.

21. A test unit according to claim 20, characterized in that it further comprises an openable and closable outlet for sucking dilution liquid from the liquid dosing chamber.

22. A test unit according to claim 21, wherein there is a common inlet/outlet connection for filling the liquid dosing chamber with dilution liquid and sucking dilution liquid from the liquid dosing chamber.

23. A test unit according to claim 22, wherein said actuator is acting on the inlet/outlet connection for said filling and sucking operations.

24. A test unit according to claim 23, wherein said actuator is a bellows, a pump or an injector.

25. A test unit according to claim 18, wherein there is a reaction chamber connected through an openable valve with the dosing chamber.

26. A test unit according to claim 18, wherein said control liquid packages are hermetically sealed collapsible bags with a diminishing volume as liquid is sucked therefrom.

27. A test unit according to claim 18, wherein said calibrator liquid and liquid reagent packages are sealed collapsible bags with a diminishing volume as liquid is sucked therefrom.

28. A test unit according to claim 18, wherein there is a refrigerator for preserving the calibrator and control liquids in frozen condition and heating means for thawing a selected calibrator or control liquid before it is sucked to the liquid dosing chamber.

29. A test unit according to claim 18, wherein the calibrator liquid is a pure component of blood serum dissolved in a solvent.

30. A test unit according to claim 18, wherein the control liquid is blood serum.

31. A system containing a plurality of test units for testing liquid samples according to claim 18, characterized in that each test unit is equipped for performing the same test reaction, that the test units are differently located and each connected through data transmission communications to a common control unit monitoring the test units in the system by data transmitted through said communications, that the reaction with the control liquid is performed in each test unit at predetermined time intervals to obtain individual control values for the test units, an average of said control values of all the test units is determined, the individual control values are compared to said average, and only test units with a reference value within a permitted deviation from the average are approved for performance of the reaction with samples to be tested.

32. A system according to claim 31, wherein in test units with a control value outside the permitted deviation from the average the reaction with the control liquid is repeated at the request of the control unit one or more times to exclude the possibility of a random error.

33. A system according to claim 31, wherein the reactions with the control liquid are performed at least daily for accepting daily test results.

34. A system containing a plurality of test units for testing liquid samples according to claim 18, characterized in that each test unit is equipped for performing the same test reaction, that the test units are differently located and each connected through data transmission communications to a common control unit monitoring the test units in the system by data transmitted through said communications, that the performance of said test system is from time to time independently evaluated by an accurate analysis of control and calibrator liquid reference samples preserved in circumstances corresponding to those prevailing in the test units, to estimate and adjust the time effect on calibrator and on control liquids and on reagents stored in the test units.
